(19) United States
(12) Patent Application Publication
(10) Pub. No.: US 2012/0116121 A1
(43) Pub. Date: May 10, 2012

(54) PROCESS FOR THE SULFOCHLORINATION OF HYDROCARBONS

(75) Inventors: Kurt F. Hirsekorn, Midland, MI (US); William Tenn, Houston, TX (US); Peter N. Nickias, Midland, MI (US)

(73) Assignee: Dow Global Technologies LLC, Midland, MI (US)

(21) Appl. No.: 13/320,773

(22) PCT Filed: Jul. 28, 2010
(86) PCT No.: PCT/US2010/043527

§ 371 Date: Nov. 16, 2011

Related U.S. Application Data
(60) Provisional application No. 61/229,863, filed on Jul. 30, 2009.

(51) Int. Cl. C07C 303/10

(52) U.S. Cl. 562/829; 562/828

(57) ABSTRACT

Produce a sulfo-chlorinated hydrocarbon using liquid sulfur dioxide, a chlorinating agent such as chlorine or sulfuryl chloride, and a metal complex catalyst, the catalyst being represented as LnM where L is at least one of an amine, phosphine, chloride or oxide, n is an integer within a range of from 1 to 6, and M is a metal selected from a group consisting of copper (Cu), ruthenium (Ru), iron (Fe), chromium (Cr), lanthanum (La), nickel (Ni), palladium (Pd), rhodium (Rh), rhenium (Re), molybdenum (Mo) and manganese (Mn).

This application is a non-provisional application claiming priority from the U.S. Provisional Patent Application No. 61/229,863, filed on Jul. 30, 2009, entitled “PROCESS FOR THE SULFOCHLORINATION OF HYDROCARBONS,” the teachings of which are incorporated by reference herein, as if reproduced in full hereinbelow.

This application relates to an improved process for sulfochlorination of hydrocarbons to produce an alkane sulfonyl chloride (for example, methane sulfonyl chloride or MSC when the hydrocarbon is methane (CH₄)).

Current processes (for example, those taught in DE 3545775, EP 194931, and EP 952147) for producing alkane sulfonyl chlorides (RSO₂Cl) from alkanes via reaction with sulfur dioxide (SO₂) and chlorine (Cl₂) typically require use of a light source, typically ultraviolet (UV) light, to at least initiate the reaction. Such light sources tend to be highly energy intensive and, accordingly, expensive.

In some aspects, this invention is a process for producing a sulfo-chlorinated hydrocarbon, which comprises a) heating a reaction mixture that comprises a hydrocarbon, a chlorinating agent selected from chlorine and sulfuryl chloride, liquid sulfur dioxide and a metal complex catalyst, the catalyst being represented as LnM where at least one ligand (L) is an amine, phosphine, chloride or oxide, n is an integer within a range of from 1 to 6, and M is at least one transition metal selected from a group consisting of copper (Cu), ruthenium (Ru), iron (Fe), chromium (Cr), lanthanum (La), nickel (Ni), palladium (Pd), rhodium (Rh), rhenium (Re), molybdenum (Mo), and manganese (Mn) and b) maintaining the reaction mixture at the reaction temperature for a period of time sufficient to convert a portion of the hydrocarbon to a sulfo-chlorinated hydrocarbon.

The transition metal is preferably selected from La, Fe, Cu, Cr and Mo. Illustrative metal complex (LₙM) catalysts include bis-diphenylphosphinoethaneiron(II) chloride ((dppe)FeCl₂); copper(I) chloride/1,1′ -dipyridyl (CuCl/2-2′bpy); chromium(III) oxide (Cr₂O₃); chromium (II) chloride (CrCl₂); chromium(III) chloride (CrCl₃); molybdenum (VI) oxide (MoO₃); and lanthanum oxide (La₂O₃).

The use of transition metal complexes (LₙM) in a condensed phase process (typically liquid sulfur dioxide) effects a desired reaction which ultimately enables propagation of free-radical sulfo-chlorination but does so with an alternate, less energy intensive and therefore less costly initiation mechanism relative to processes that use light for initiation,

The process occurs with SO₂ in a condensed or liquid phase. Alternate solvents include concentrated hydrochloric acid (HCl), carbon tetrachloride (CCl₄) or a mixture of either or both with liquid SO₂.

Run the process with chlorine as a limiting reagent relative to the hydrocarbon and sulfur dioxide. Preferably, maintain both a hydrocarbon to chlorine ratio and a sulfur dioxide to chlorine ratio above 1.

In the above process, bring the reaction mixture to a temperature sufficient to effect a reaction among reaction mixture components. The temperature is suitably within a range of from 80° C. to 110° C. Maintain the temperature for a period of time sufficient to achieve a desired yield of sulfo-chlorinated hydrocarbon. Suitable periods of time range from two hours to 20 hours.

The hydrocarbon is selected from alkanes (for example, methane, ethane, and propane) and alkenes with a suitably reactive carbon-hydrogen bond (for example, propylene, butene and hexene). A particularly desirable sulfo-chlorinated hydrocarbon is methane sulfonyl chloride.

The chlorinating agent is selected from chlorine and sulfuryl chloride (SO₂Cl₂) or a mixture thereof, with chlorine alone providing very satisfactory results in terms of yield of alkane sulfonyl chloride. Alternate chlorinating agents include trifluoro-methane sulfonyl chloride (CF₃SO₂Cl) and methane sulfonyl chloride (CH₃SO₂Cl).

### EXAMPLE

### Example 1

Use a 100 milliliter (mL) Hastelloy™ C, agitated reactor (Parr Instruments) to effect sulfochlorination of methane using methane (CH₄) and catalyst (bis-diphenylphosphinoethaneiron(II) chloride (dppe)FeCl2)) loadings (in millimoles (mmol)) as shown in Table 1 below. Load catalyst into the reactor, seal the reactor, cool reactor contents to −10 degrees centigrade (° C.) and then charge and condense approximately 20 grams (g) (312 millimoles (mmol)) of SO₂ into the reactor. Charge 20 pounds per square inch (psi) of Cl₂ (5.3 mmol) and 190 psi of methane (51.4 mmol) to the reactor, then heat reactor contents to a reaction temperature as shown in Table 1 and maintain reactor contents at that temperature for a period of time (in hours (h)) also as shown in Table 1. Allow reactor contents to cool to room temperature (nominally 25° C.), then take a 1 liter (L) sample of the reactor's gas phase for analysis by gas chromatography (GC) before venting remaining gaseous components to a caustic scrubber. Measure and analyze liquid contents of the reactor via GC, proton (¹H) nuclear magnetic resonance (NMR) spectroscopy and carbon 13 (¹³C) NMR spectroscopy. For NMR analyses, add a known amount of chloroform-d-containing cyclohexane as an internal standard. Calculate percent yield (percent yield) as moles of MSC produced divided by moles of initial Cl₂ added. In Table 1, RH refers to hydrocarbon (CH₄, C₃H₈ (propane) or C₂H₆ (ehane)) and RSC refers to sulfochlorinated hydrocarbon.

### Examples 2-11 and Comparative Examples (CEx) A-E

Replicate of Ex 1 with changes in catalyst and, for Ex 10 and 11, hydrocarbon as shown in Table 1 below,

TABLE 1

| | | RH | | Catalyst | RSC | % Yield, | | |
|---|---|---|---|---|---|---|---|---|
| Ex/ | | Loading | | (loading) | Produced | based on | Temp. | Time |
| CEx | RH | (mmol) | Catalyst | (mmol) | (mmol) | Cl₂ | (° C.) | (h) |
| 1 | CH₄ | 51.4 | (dppe)FeCl₂ | 0.074 | 0.487 | 9.2 | 100 | 14 |
| 2 | CH₄ | 75.9 | CuCl/2bpy | 1.57 | 0.131 | 1.5 | 80 | 6 |
| 3 | CH₄ | 75.9 | (dppe)FeCl₂ | 0.3 | 0.201 | 3.8 | 80 | 20 |
| A | CH₄ | 93.25 | (Ph₃P)₃RuCl₂ | 0.03 | 0.1 | 0.01 | 80 | 20 |
| 4 | CH₄ | 51 | Cr₂O₃ | 0.34 | 1.7 | 32.8 | 80 | 2 |
| 5 | CH₄ | 51 | CrCl₂ | 0.3 | 0.5 | 10 ± 2 | 80 | 2 |
| 6 | CH₄ | 51 | CrCl₂ | 0.8 | 0.95 | 18 | 80 | 2 |
| B | CH₄ | 51 | CrCl₃ | 0.5 | 0 | 0 | 80 | 2 |
| 7 | CH₄ | 51 | Cr₂O₃ | 0.3 | 2.6 | 47 ± 9^{b} | 110 | 2 |
| C | CH₄ | 51 | V₂O₃ | 0.29 | 0 | 0 | 80 | 2 |
| 8 | CH₄ | 51 | MoO₃ | 0.29 | 0.47 | 9 ± 4 | 80 | 2 |
| 9 | CH₄ | 51 | La₂O₃ | 0.29 | 0.64 | 12 ± 2 | 80 | 2 |
| D | CH₄ | 51 | Fe₂O₃ | 0.3 | 0.005 | <1 | 80 | 2 |
| E | CH₄ | 51 | CuO | 0.4 | 0 | 0 | 80 | 2 |
| 10 | C₃H₈ | 26 | Cr₂O₃ | 0.3 | 4.2 | 79 ± 5 | 80 | 1 |
| 11 | C₂H₆ | 43 | Cr₂O₃ | 0.3 | 1.9 | 37 ± 7 | 80 | 1 |

The data summarized in Table 1 represent evaluations of a number of materials as potential catalysts for hydrocarbon sulfochlorination. CEx B, C and E show no MSC production under reaction conditions shown in Table 1 with, respectively, chromium (III) chloride (CrCl₃), vanadium oxide (V₂O₃) and copper oxide (CuO). CEx A and CEx D show very little (less than 1 percent) MSC production under reaction conditions shown in Table 1 with, respectively triphenylphosphine ruthenium chloride ((Ph₃P)₃Rul₂) and ferric oxide (Fe₂O₃). By way of contrast, chromium (II) chloride (CrCl₂) (Ex 5 and 6), chromium oxide (Cr₂O₃) (Ex 4 and 7), molybdenum oxide (MoO₃) (Ex 8) and lanthanum oxide (La₂O₃) all show MSC yields of approximately 10 percent or more, at least a tenfold increase over CEx A and D. Ex 1 and 2 show how reaction conditions affect MSC yield using (dppe)FeCl₂ as catalyst. Ex 3 shows low (1.5 percent) yield with CuCl/2,2′-bpy as catalyst under reaction conditions shown in Table 1. Ex 10 (sulfochlorination of propane) and Ex 11 (sulfochlorination of ethane) show very good RSC yields with Cr₂O₃ as a catalyst. Although not shown here, control experiments under similar conditions to those shown in Table 1, but with no catalyst, also yield no MSC.

1. A process for producing a sulfo-chlorinated hydrocarbon, which process comprises a) heating a reaction mixture that comprises a hydrocarbon, a chlorinating agent selected from chlorine and sulfuryl chloride, liquid sulfur dioxide and a metal complex catalyst, the catalyst being represented as LnM where L is at least one of an amine, phosphine, chloride or oxide, n is an integer within a range of from 1 to 6, and M is at least one metal selected from a group consisting of copper (Cu), ruthenium (Ru), iron (Fe), chromium (Cr), lanthanum (La), nickel (Ni), palladium (Pd), rhodium (Rh), rhenium (Re), molybdenum (Mo) and manganese (Mn) to a reaction temperature, and b) maintaining the reaction mixture at the reaction temperature for a period of time sufficient to convert a portion of the hydrocarbon to a sulfo-chlorinated hydrocarbon.

2. The process of claim 1, wherein the temperature is within a range of from 80 degrees centigrade to 110 degrees centigrade.

3. The process of claim 1, wherein the period of time is within a range of from two hours to 20 hours.

4. The process of claim 1 where the transition metal is preferably selected from La, Fe, Cu, Cr and Mo.

5. The process of claim 1, wherein the catalyst is selected from a group consisting of bis-diphenylphosphinoethane iron (II) chloride, copper (I) chloride/1,1′-dipyridyl, chromium (III) oxide, chromium (II) chloride, molybdenum (VI) oxide, and lanthanum oxide.

6. The process of claim 1, wherein the hydrocarbon is selected from alkanes and alkenes with a reactive carbon hydrogen bond.

7. The process of claim 6, wherein the hydrocarbon is selected from methane, ethane, propane.

8. The process of claim 1, wherein the hydrocarbon is methane and the sulfo-chlorinated hydrocarbon is methane sulfonyl chloride.
